(19) United States
(12) Patent Application Publication
(10) Pub. No.: US 2022/0362126 A1
(43) Pub. Date: Nov. 17, 2022

(54) COSMETIC COMPOSITION AND METHOD

(71) Applicant: Mark A. Foglietti, Beachwood, OH (US)

(72) Inventor: Mark A. Foglietti, Beachwood, OH (US)

(21) Appl. No.: 17/744,257

(22) Filed: May 13, 2022

Related U.S. Application Data
(60) Provisional application No. 63/188,448, filed on May 13, 2021.

(51) Int. Cl. A61K 8/66; A61K 8/46; A61K 8/73; A61K 8/14; A61K 8/02; A61Q 19/08

(52) U.S. Cl. CPC A61K 8/66; A61K 8/46; A61K 8/735; A61K 8/14; A61K 8/0208; A61Q 19/08; A61K 2800/56; A61K 2800/49; A61K 2800/546

(57) ABSTRACT

A cosmetic composition includes one or more solvents and one or more rejuvenation cosmetic components. The one or more solvents include dimethyl sulfoxide and/or dimethyl sulfone. The one or more rejuvenation cosmetic components, include botulinum toxin, hyaluronic acid, and/or exosomes. A delivery system includes a transdermal patch including the cosmetic composition. The cosmetic composition is topically applied to the skin of a patient.

### RELATED APPLICATION DATA

This application claims the benefit of U.S. Provisional Patent Application No. 63/188,448, filed May 13, 2021, the disclosure of which is incorporated herein by reference in its entirety.

### TECHNICAL FIELD

The present disclosure relates to a cosmetic composition, and more specifically to a cosmetic composition that can be used for the transcutaneous delivery of one or more rejuvenation cosmetic components, such as botulinum toxin, hyaluronic acid, and/or exosomes.

### BACKGROUND

Conventionally, rejuvenation cosmetic components such as botulinum toxin, hyaluronic acid, and exosomes are administered to a patient using an invasive injection or procedure. Injections carry the risk of pain, bruising, bleeding, and infection, and repetitive injections at the same site can result in scarring and deformity. Injections also provide a point-source for application, and it can be difficult or impossible to achieve a desired cosmetic effect in areas where a uniform application is desired. Moreover, there are areas on the body where needle injection is not preferred. Accordingly, there remains a need for further contributions in this area of technology.

### SUMMARY OF THE INVENTION

This application relates to a cosmetic composition that can be used for the transcutaneous delivery of one or more rejuvenation cosmetic components, such as botulinum toxin, hyaluronic acid, and/or exosomes. The transcutaneous delivery of these cosmetic components using the cosmetic composition of the present disclosure may allow for delivery to target tissue without the use of needles or invasive procedure, and provides a significant advance in rejuvenation medicine.

In accordance an aspect of the present disclosure, a cosmetic composition includes: one or more solvents, the one or more solvents comprising dimethyl sulfoxide and/or dimethyl sulfone; and one or more rejuvenation cosmetic components, the one or more rejuvenation cosmetic components comprising botulinum toxin, hyaluronic acid, and/or exosomes.

In some embodiments, the one or more solvents is dimethyl sulfoxide.

In some embodiments, the one or more solvents is dimethyl sulfone.

In some embodiments, the one or more solvents is a combination of dimethyl sulfoxide and dimethyl sulfone.

In some embodiments, a total amount of the one or more solvents in the cosmetic composition ranges from 0.1 vol % to 99.9 vol %.

In some embodiments, a total amount of the one or more solvents in the cosmetic composition ranges from 0.1 vol % to 50 vol %.

In some embodiments, the one or more rejuvenation cosmetic components includes botulinum toxin.

In some embodiments, the one or more rejuvenation cosmetic components includes hyaluronic acid.

In some embodiments, the one or more rejuvenation cosmetic components includes exosomes.

In some embodiments, the one or more rejuvenation cosmetic components includes a combination of botulinum toxin and hyaluronic acid.

In some embodiments, the one or more rejuvenation cosmetic components includes a combination of botulinum toxin and exosomes.

In some embodiments, the one or more rejuvenation cosmetic components includes a combination of hyaluronic acid and exosomes.

In some embodiments, the one or more rejuvenation cosmetic components includes a combination of botulinum toxin, hyaluronic acid, and exosomes.

In some embodiments, the cosmetic composition further includes water.

In some embodiments, the cosmetic composition further includes one or more oils.

In some embodiments, the cosmetic composition further includes one or more water-soluble or water-swelling polymers.

In some embodiments, the cosmetic composition is a liquid composition in the form of a cream, paste, gel, or mousse.

In accordance with another aspect of the present disclosure, a delivery system includes a transdermal patch comprising the cosmetic composition.

In some embodiments, the transdermal patch includes: a backing layer; a rate-controlling membrane layer; a drug reservoir disposed between the backing layer and the rate-controlling membrane layer, the drug reservoir containing the cosmetic composition; and an adhesive layer adjacent a surface of the rate-controlling membrane layer distal the drug reservoir.

In some embodiments, the transdermal patch includes: a backing layer; and a drug and adhesive layer adjacent the backing layer, the drug and adhesive layer comprising a matrix of adhesive in which the cosmetic composition is disposed.

In accordance with another aspect of the present disclosure, a method of transcutaneous delivery of one or more rejuvenation cosmetic components includes topically applying a therapeutically effective amount of the cosmetic composition to the skin of a patient

The following description sets forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

FIG. 1 is a schematic cross-sectional view showing layers of an exemplary transdermal patch.

FIG. 2 is a schematic cross-sectional view showing layers of an exemplary transdermal patch.

### DETAILED DESCRIPTION

All ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The transitional words or phrases, such as “comprising,” “including,” “carrying,” “having,” “containing,” “involving,” “holding,” and the like, are to be understood to be open-ended, i.e., to mean including but not limited to.

Embodiments of the cosmetic composition of the present disclosure include one or more solvents and one or more rejuvenation cosmetic components.

Examples of solvents suitable for use in the composition include dimethyl sulfoxide (DMSO), dimethyl sulfone (DMSO₂), and combinations thereof. The solvents in the cosmetic composition can be provided in any suitable amount. In some embodiments, the total amount of the one or more solvents in the cosmetic composition ranges from 0.01 vol % to 99.9 vol %. In some embodiments, the total amount of the one or more solvents in the cosmetic composition ranges from 0.1 vol % to 99.9 vol %. In some embodiments, the total amount of the one or more solvents in the cosmetic composition ranges from 0.1 vol % to 90 vol %. In some embodiments, the total amount of the one or more solvents in the cosmetic composition ranges from 0.1 vol % to 75 vol %. In some embodiments, the total amount of the one or more solvents in the cosmetic composition ranges from 25 vol % to 75 vol %. In some embodiments, the total amount of the one or more solvents in the cosmetic composition ranges from 0.1 vol % to 50 vol %. In some embodiments, the total amount of the one or more solvents in the cosmetic composition ranges from 1 vol % to 50 vol %. In some embodiments, the total amount of the one or more solvents in the cosmetic composition ranges from 25 vol % to 50 vol %. In some embodiments, the total amount of the one or more solvents in the cosmetic composition ranges from 1 vol % to 20 vol %. In some embodiments, the total amount of the one or more solvents in the cosmetic composition ranges from 1 vol % to 10 vol %. In some embodiments, the total amount of the one or more solvents in the cosmetic composition ranges from 1 vol % to 5 vol %.

In those embodiments in which both DMSO and DMSO₂ are included as solvents in the cosmetic composition, the amount of DMSO in the total amount of the one or more solvents may range from 0.1 vol % to 99.9 vol % and the amount of DMSO₂ in the total amount of the one or more solvents ranges from 0.1 vol % to 99.9 vol %. In some embodiments, the amount of DMSO in the total amount of the one or more solvents ranges from 25 vol % to 75 vol % and the amount of DMSO₂ in the total amount of the one or more solvents ranges from 25 vol % to 75 vol %. In some embodiments, the amount of DMSO in the total amount of the one or more solvents ranges from 40 vol % to 60 vol % and the amount of DMSO₂ in the total amount of the one or more solvents ranges from 40 vol % to 60 vol %.

The one or more solvents may act as a vehicle for the one or more rejuvenation cosmetic components of the composition by effectuating transcutaneous delivery of the one or more rejuvenation cosmetic components to target tissue of a patient. For example, the cosmetic composition utilizing DMSO or DMSO₂ may enhance the delivery of the known active drugs and particles into and through the skin.

Exemplary rejuvenation cosmetic components include botulinum toxin, hyaluronic acid, and exosomes. It will be appreciated, however, that other rejuvenation cosmetic components can be used in the cosmetic composition.

Botulinum toxin is a neurotoxic protein produced by bacterium that prevents the release of the neurotransmitter, acetylcholine from the axon endings at the neuromuscular junction, thus causing flaccid paralysis, or in the case of cosmetic use, the relaxation of the targeted muscles that cause skin lines and wrinkles. This medication conventionally requires a skin injection in order to deliver it to the targeted facial or body muscles.

Hyaluronic acid is a substance naturally produced in the body and found in the skin and connective tissue. Its main function is to retain water and keep tissues well lubricated. The medical synthesized product is a complex sugar that is in gel form. This refined product is injected into the lines and grooves of the skin so as to plump or soften a line or groove in the skin or plump the tissue such as the lips. This medication also conventionally requires the use of an injection needle syringe system and direct placement of the medication into the specified soft tissue and skin.

Exosomes are extracellular vesicles, EV's, that are released by cells in order to communicate with one another utilizing genetic information, mRNA and micro mRNA. This communication theoretically can signal a cell to rejuvenate, depending upon the type of exosome utilized. Conventionally, exosomes are delivered to the targeted area by direct injection with a needle. IV infusion, or micro needling of the skin in order to get penetration into the skin surface and deeper.

The amount and concentration of each of the one or more rejuvenation cosmetic components can be provided in any suitable amount in the cosmetic composition. The amount and concentration thereof for effective therapeutic treatment can vary, depending on the size and area of the patient to be treated. In some examples, the botulinum toxin of the cosmetic composition is provided at 1 unit to 200 units. In other examples, the botulinum toxin of the cosmetic composition is provided at 1 unit to 100 units. In other examples, the botulinum toxin of the cosmetic composition is provided at 1 unit to 50 units. In some examples, the hyaluronic acid is provided in an amount ranging from 0.1 mg/mL to 100 mg/mL. In other examples, the hyaluronic acid is provided in an amount ranging from 0.1 mg/mL to 50 mg/mL. In other examples, the hyaluronic acid is provided in an amount ranging from 1 mg/mL to 25 mg/mL. In other examples, the hyaluronic acid is provided in an amount ranging from 5 mg/mL to 20 mg/mL. In some examples, the exosomes is provided in an amount ranging from 0.1 μg/mL to 1000 μg/mL. In other examples, the exosomes is provided in an amount ranging from 1 μg/mL to 500 μg/mL. In other examples, the exosomes is provided in an amount ranging from 1 μg/mL to 100 μg/mL. In other examples, the exosomes is provided in an amount ranging from 10 μg/mL to 50 μg/mL.

The one or more rejuvenation cosmetic component(s) can be combined directly with the one or more solvents. In other embodiments, one or more rejuvenation cosmetic component(s) can be provided in solution, which is combined with the one or more solvents. In solution can constitute the rejuvenation cosmetic component being provided in liquid or gel. Exemplary, non-limiting, solutions include water, saline, solvent, gelatin, and the like. In some embodiments, the total amount of the rejuvenation cosmetic component(s) provided in solution in the composition ranges from 0.1 vol % to 99.9 vol %. In some embodiments, the total amount of the rejuvenation cosmetic component(s) provided in solution in the composition ranges from 0.1 vol % to 95 vol %. In some embodiments, the total amount of the rejuvenation cosmetic component(s) provided in solution in the composition ranges from 1 vol % to 95 vol %. In some embodiments, the total amount of the rejuvenation cosmetic component(s) provided in solution in the composition ranges from 5 vol % to 95 vol %. In some embodiments, the total amount of the rejuvenation cosmetic component(s) provided in solution in the composition ranges from 15 vol % to 95 vol %. In some embodiments, total amount of the rejuvenation cosmetic component(s) provided in solution in the composition ranges from 25 vol % to 95 vol %. In some embodiments, the total amount of the rejuvenation cosmetic component(s) provided in solution in the composition ranges from 40 vol % to 95 vol %. In some embodiments, the total amount of the rejuvenation cosmetic component(s) provided in solution in the composition ranges from 50 vol % to 95 vol %. In some embodiments, the total amount of the rejuvenation cosmetic component(s) provided in solution in the composition ranges from 50 vol % to 90 vol %. In some embodiments, the total amount of the rejuvenation cosmetic component(s) provided in solution in the composition ranges from 60 vol % to 90 vol %. In some embodiments, the total amount of the rejuvenation cosmetic component(s) provided in solution in the composition ranges from 25 vol % to 75 vol %. In some embodiments, the total amount of the rejuvenation cosmetic component(s) provided in solution in the composition ranges from 25 vol % to 50 vol %. In some embodiments, the total amount of the rejuvenation cosmetic component(s) provided in solution in the composition ranges from 1 vol % to 50 vol %. In some embodiments, the total amount of the rejuvenation cosmetic component(s) provided in solution in the composition ranges from 5 vol % to 50 vol %. In some embodiments, the total amount of the rejuvenation cosmetic component(s) provided in solution in the composition ranges from 1 vol % to 25 vol %.

In some embodiments, the one or more rejuvenation cosmetic components includes at least one of botulinum toxin, hyaluronic acid, and exosomes. In some embodiments, the cosmetic includes more than one rejuvenation cosmetic component.

In those embodiments in which more than one rejuvenation cosmetic component provided in solution is included in the composition, the amount of each rejuvenation cosmetic component provided in solution in the total amount of the one or more rejuvenation cosmetic components provided in solution may range from 0.1 vol % to 99.9 vol %. For example, in embodiments in which botulinum toxin provided in solution and hyaluronic acid provided in solution are included, the amount of botulinum toxin provided in solution in the total amount of the one or more rejuvenation cosmetic components provided in solution ranges from 0.1 vol % to 99.9 vol % and the amount of hyaluronic acid provided in solution in the total amount of the one or more rejuvenation cosmetic components provided in solution ranges from 0.1 vol % to 99.9 vol %. In embodiments in which botulinum toxin provided in solution and exosomes provided in solution are included, the amount of botulinum toxin provided in solution in the total amount of the one or more rejuvenation cosmetic components provided in solution ranges from 0.1 vol % to 99.9 vol % and the amount of exosomes provided in solution in the total amount of the one or more rejuvenation cosmetic components provided in solution ranges from 0.1 vol % to 99.9 vol %. In embodiments in which hyaluronic acid provided in solution and exosomes provided in solution are included, the amount of hyaluronic acid provided in solution in the total amount of the one or more rejuvenation cosmetic components provided in solution ranges from 0.1 vol % to 99.9 vol % and the amount of exosomes provided in solution in the total amount of the one or more rejuvenation cosmetic components provided in solution ranges from 0.1 vol % to 99.9 vol %. In embodiments in which botulinum toxin provided in solution, hyaluronic acid provided in solution, and exosomes provided in solution are included, the amount of botulinum toxin provided in solution in the total amount of the one or more rejuvenation cosmetic components provided in solution ranges from 0.1 vol % to 99.9 vol %, the amount of hyaluronic acid provided in solution in the total amount of the one or more rejuvenation cosmetic components provided in solution ranges from 0.1 vol % to 99.9 vol %, and the amount of exosomes provided in solution in the total amount of the one or more rejuvenation cosmetic components provided in solution ranges from 0.1 vol % to 99.9 vol %.

The combination of the one or more rejuvenation cosmetic components with the one or more solvents (e.g., DMSO and/or DMSO₂) may provide for the percutaneous penetration of the medication of skin to the target tissue without the need for needle sticks or invasive procedure and the associated trauma. As such, a cosmetic composition of the present disclosure including botulinum toxin may provide, for example, relaxation of one or more target muscles. A cosmetic composition of the present disclosure including hyaluronic acid may provide, for example, hydration of skin in a target area associated with skin lines. A cosmetic composition of the present disclosure including exosomes may provide, for example, rejuvenation of target skin cells. And to the extent that more than one rejuvenation cosmetic component is provided in the cosmetic composition, a plurality of such effects may be provided.

The form of the cosmetic composition is not particularly limited. Exemplary forms include a liquid, emulsion, cream, paste, gel, mousse, and the like. In some embodiments, the cosmetic composition can be provided as part of a delivery system, such as a transdermal patch.

Various factors can affect the absorption rates and penetration depth of topical pharmaceutical preparations, including the nature of the active ingredient, the nature of the vehicle, the pH and the relative solubility of the vehicle versus the skin. (J of Pharmaceutical Sciences, 60: 1175-1179 (1979). As such, the relative amounts of the one or more solvents and the one or more cosmetic components can be varied depending on one or more factors such as the particular cosmetic component(s) used, the form of the cosmetic composition (e.g., liquid, gel, cream, spray, etc.), the particular application site, etc.

Depending on the form of the cosmetic, the cosmetic composition may comprise one or more additional components. Examples of additional components include water, one or more water-soluble or water-swelling polymers, and one or more oils.

In embodiments in which water is included as a constituent component of the cosmetic composition, the water content may range from 0.1 vol % to 99.0 vol %. In some embodiments in which water is included as a constituent component of the cosmetic composition, the water content may range from 0.1 vol % to 90.0 vol %. In some embodiments in which water is included as a constituent component of the cosmetic composition, the water content may range from 0.1 vol % to 50.0 vol %.

Examples of the water-soluble or water-swelling polymer include plant polymers such as gum Arabic, tragacanth gum, arabinogalactan, locust bean gum (carob gum), guar gum, karaya gum, carrageenan, pectin, agar, quince seed (i.e., marmelo), starch from rice, corn, potato or wheat, algae colloid, and trant gum; bacteria-derived polymers such as xanthan gum, dextran, succinoglucan, and pullulan; animal-derived polymers such as collagen, casein, albumin, and gelatin; starch-derived polymers such as carboxymethyl starch and methylhydroxypropyl starch; cellulose polymers such as methyl cellulose, ethyl cellulose, methylhydroxypropyl cellulose, carboxymethyl cellulose, hydroxymethyl cellulose, hydroxypropyl cellulose, nitrocellulose, sodium cellulose sulfate, sodium carboxymethyl cellulose, crystalline cellulose, and cellulose powder; alginic acid-derived polymers such as sodium alginate and propylene glycol alginate; vinyl polymers such as polyvinyl methylether, and carboxyvinyl polymer; polyoxyethylene polymers; polyoxyethylene/polyoxypropylene copolymers; acrylic polymers such as sodium polyacrylate, polyethyl acrylate, and polyacrylamide; polyethyleneimine; cationic polymers; and water-soluble inorganic polymers such as, bentonite, aluminum magnesium silicate, laponite, hectorite, and silicic anhydride. Film forming agents, such as polyvinyl alcohol and polyvinyl pyrrolidone, are also included. In embodiments in which one or more water-soluble or water-swelling polymers is included as a constituent component of the cosmetic composition, the total content of water-soluble or water-swelling polymers may range from 0.1 vol % to 70.0 vol %. In some embodiments in which one or more water-soluble or water-swelling polymers is included as a constituent component of the cosmetic composition, the total content of water-soluble or water-swelling polymers may range from 0.1 vol % to 50.0 vol %. In some embodiments in which one or more water-soluble or water-swelling polymers is included as a constituent component of the cosmetic composition, the total content of water-soluble or water-swelling polymers may range from 0.1 vol % to 30.0 vol %.

The one or more oils agent may be liquid, semi-solid, or solid at an ambient temperature. Exemplary oils include natural fats and oils from animals and plants, semi-synthetic oils, hydrocarbon oils, higher fatty acids, higher alcohols, ester oils, glyceride oils, silicone oils, and flouorinated oils.

Examples of natural fats and oils from animals and plants and semi-synthetic oils include avocado oil, linseed oil, almond oil, Ibota wax, perilla oil, olive oil, cacao butter, kapok wax, kaya oil, carnauba wax, Glycyrrhiza oil, candelilla wax, beef tallow, neat's-foot oil, beef bone fat, hydrogenated beef tallow, apricot kernel oil, spermaceti wax, hydrogenated oil, wheat germ oil, sesame oil, rice germ oil, rice bran oil, sugar cane wax, sasanqua oil, safflower oil, shear butter, Chinese tung oil, cinnamon oil, jojoba wax, shellac wax, turtle oil, soybean oil, tea seed oil, camellia oil, evening primrose oil, corn oil, lard, rapeseed oil, Japanese tung oil, rice bran oil, germ oil, horse fat, persic oil, palm oil, palm kernel oil, castor oil, hydrogenated castor oil, castor oil fatty acidmethyl ester, sunflower oil, grape oil, bayberry wax, jojoba oil, macadamia nut oil, beeswax, mink oil, cottonseed oil, cotton wax, Japanese wax, Japanese wax kernel oil, montan wax, coconut oil, hydrogenated coconut oil, tri-coconut oil fatty acid glyceride, mutton tallow, peanut oil, lanolin, liquid lanolin, hydrogenated lanolin, lanolin alcohol, hard lanolin, lanolin acetate, isopropyl lanolate, hexyl laurate, POE lanolin alcohol ether, POE lanolin alcohol acetate, polyethylene glycol lanolate, POE hydrogenated lanolin alcohol ether, and egg yolk oil.

Examples of the hydrocarbon oils include ozokerite, squalane, squalene, ceresin, paraffin, paraffin wax, liquid paraffin, pristane, polyisobutylene, microcrystalline wax, and Vaseline.

Examples of the higher fatty acids include lauric acid, myristic acid, palmitic acid, stearic acid, behenic acid, undecylenic acid, oleic acid, linoleic acid, linolenic acid, arachidonic acid, eicosapentaenoic acid (EPA), docosahexaenoic acid (DHA), isostearic acid, and 12-hydroxystearic acid.

Examples of the higher alcohols include lauryl alcohol, myristyl alcohol, palmityl alcohol, stearyl alcohol, behenyl alcohol, hexadecyl alcohol, oleyl alcohol, isostearyl alcohol, hexyldodecanol, octyl dodecanol, cetostearyl alcohol, 2-decyltetradecinol, cholesterol, phytosterol, sitosterol, lanosterol, POE cholesterol ether, POE phytosterol ether, monostearyl glycerin ether (batyl alcohol), and monooleyl glyceryl ether (cerakyl alcohol), etc.

Examples of the ester oils include diisobutyl adipate, 2-hexyldecyl adipate, di-2-heptylundecyl adipate, N-alkyl glycol monoisostearate, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprirate, triethyl citrate, 2-ethylhexyl succinate, amyl acetate, ethyl acetate, butyl acetate, isocetyl stearate, butyl stearate, diisopropyl sebacinate, di-2-ethylhexyl sebacinate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid esters, isopropyl myristate, octyldodecyl myristate, 2-hexyldecyl myristate, myristyl myristate, hexyldecyl dimethyloctanoate, ethyl laurate, hexyl laurate, 2-octyldodecyl N-lauroyl-L-glutamate, and diisostearyl malate.

Examples of the glyceride oils include acetoglyceryl, glycerol triisooctanoate, glyceryl triisostearate, glyceryl triisopalmitate, glyceryl monostearate, glyceryl di-2-heptylundecanoate, glyceryl trimyristate, and di-glyceryl myristate isostearate.

Examples of the silicone oils include organopolysiloxanes which have a low to high viscosity and are liquid at ambient temperatures, such as dimethylpolysiloxane, methylphenylpolysiloxane, methylhydrogenpolysiloxane, dimethylsiloxane/methylphenylsiloxane copolymer; cyclic siloxane such as octamethylcyclotetrasiloxane, decamethylcyclopentasiloxane, dodecamethylcyclohexasiloxane, tetramethyltetrahydrogencyclotetrasil-oxane, tetramethyltetraphenylcyclotetrasiloxane; branched siloxane such as tristrimethylsiloxysilane, tetrakistrimethylsiloxysilane, tristrimethylsiloxyphenylsilane; higher alkoxy-modified silicone such as stearoxy silicone, alkyl-modified silicone, amino-modified silicone, and fluorine-modified silicone.

Examples of the fluorine-modified oils include perfluoropolyethers, perfluorodecaline, perfluorooctane, fluorinated pitch, and fluoroalcohols and one or two or more of these may be used as required.

In embodiments in which one or more oils is included as a constituent component of the cosmetic composition, the total content of oils may range from 0.1 vol % to 99 vol %. In some embodiments in which one or more oils is included as a constituent component of the cosmetic composition, the total content of oils may range from 0.1 vol % to 90 vol %. In some embodiments in which one or more oils is included as a constituent component of the cosmetic composition, the total content of oils may range from 0.1 vol % to 50 vol %. In some embodiments in which one or more oils is included as a constituent component of the cosmetic composition, the total content of oils may range from 0.1 vol % to 30 vol %.

Examples of other additional components include oil-soluble gelling agents, clay minerals modified with organic compounds, moisture retention agents, antiseptics, anti-bacterial agents, fragrances, salts, antioxidants, pH regulators, chelating agents, refreshing agents, anti-inflammatory agents, skin beautifying components, such as skin whitener, cell activator, rough dry skin improver, blood circulation promoter, skin astringent and anti-seborrheic agent, vitamins, amino acids, nucleic acids, hormones, and clathrate compounds. The one or more additional components may be provided in the cosmetic composition in any suitable amount.

The oil-soluble gelling agent may be selected from metal soaps such as aluminum stearate, magnesium stearate, and zinc myristate; α-amino acid derivatives such as N-lauroyl-L-glutamic acid, α, γ-di-n-butylamine; dextrin fatty acid esters such as dextrin palmitate, dextrin stearate, and dextrin 2-ethylhexane palmitate; sucrose fatty acid esters such as sucrose palmitate and sucrose stearate; benzylidene derivatives of sorbitol such as monobenzylidene sorbitol and dibenzylidene sorbitol; clay minerals modified with an organic moiety such as dimethylbenzyldodecylammonium montmorillonite clay, dimethyldioctadecylammonium montmorillonite, and one or two or more gelling agents may be used, if necessary.

Examples of the moisture retention agents include glycerin, sorbitol, propylene glycol, dipropylene glycol, 1,3-butylene glycol, pentylene glycol, glucose, xylitol, maltitol, polyethylene glycol, hyaluronic acid, chondroitin sulfuric acid, pyrrolidone carboxylate, polyoxyethylene methylglycoside, and polyoxypropylene methylglycoside.

Examples of the antiseptics indluce alkyl paraoxybenzoates, benzoic acid, sodium benzoate, sorbic acid, potassium sorbate, and phenoxyethanol may be used. For the antibacterial agents, benzoic acid, salicylic acid, carbolic acid, sorbic acid, paraoxybenzoic acid alkyl esters, parachloromethacresol, hexachlorophene, benzalkonium chloride, chlorohexydine chloride, trichlorocarbanilide, triclosan, photosensitizer, and phenoxyethanol.

Examples of the antioxidants include tocopherol, butylhydroxyanisole, dibutylhydroxytoluene and phytic acid; examples of the pH regulators include lactic acid, citric acid, glycolic acid, succinic acid, tartaric acid, dl-malic acid, potassium carbonate, sodium hydrogen carbonate and ammonium hydrogen carbonate; examples of the chelating agents include alanine, sodium ethylenediaminetetraacetate, sodium polyphosphate, sodium metaphosphate and phosphoric acid; examples of the refrigerants include L-menthol and camphor; and examples of the anti-inflammatory agents include allantoin, glycyrrhetinic acid, glycyrrhizinic acid, tranexamic acid, and azulene.

Examples of the skin-beautifying components include whitening agents, such as placenta extract, arbutin, glutathione and Yukinoshita extract; cell activators, such as royal jelly, photosensitizers, cholesterol derivatives and calf blood extract; rough and dry skin improvers; blood circulation improvers, such as nonylic acid vanillyl amide, benzyl nicotinate, beta-butoxyethyl nicotinate, capsaicin, zingerone, cantharis tincture, ichtammol, caffeine, tannic acid, alpha-borneol, tocopheryl nicotinate, inositol hexanicotinate, cyclandelate, cinnarizine, tolazoline, acetyl choline, verapamil, cepharanthin and gamma-oryzanol; skin astringents, such as zinc oxide and tannic acid; and anti-seborrheic agents, such as sulfur and thianthol; vitamins, e.g. vitamin A such as vitamin A oil, retinol, retinyl acetate and retinyl palmitate; vitamin B₂ such as riboflavin, riboflavin butyrate and flavin adenine nucleotide, vitamin B₆ such as pyridoxine hydrochloride, pyridoxine dioctanoate and pyridoxine tripalmitate, vitamin B₁₂ and its derivatives, and vitamin B₁₅ and its derivatives; vitamin C, such as L-ascorbic acid, L-ascorbate dipalmitate, sodium (L-ascorbic acid)-2-sulfate and dipotassium L-ascorbic acid diphosphate; vitamin D, such as ergocalciferol and cholecarciferol; vitamin E, such as alpha-tocopherol, beta-tocopherol, gamma-tocopherol, dl-alpha-tocopheryl acetate, dl-alpha-tocopheryl nicotinate and dl-alpha-tocopheryl succinate; vitamin H; vitamin P; nicotinic acids, such as nicotinic acid, benzyl nicotinate and nicotinic acid amide; pantothenic acids, such as calcium pantothenate, D-pantothenyl alcohol, pantothenyl ethyl ether and acetylpantothenyl ethyl ether; aloe; and biotin.

Examples of the amino acids include glycine, valine, leucine, isoleucine, serine, threonine, phenylaranine, alginine, lysine, aspartic acid, glutamic acid, cystine, cysteine, methionine, and tryptophan; examples of the nucleic acids include deoxyribonucleic acid; and examples of the hormones include estradiol and ethenyl estradiol.

In producing the cosmetic composition of the present disclosure, the components (e.g., one or more solvents, one or more rejuvenation cosmetic components, and optionally one or more additional components) can be combined and mixed so as to form the composition. The produced cosmetic composition can be stored in an appropriate container prior to use. In embodiments in which the cosmetic composition is to be included as part of a delivery system such as a transdermal patch, the cosmetic composition can be applied to a carrier layer or contained in a drug reservoir of the delivery system.

In a method of treatment, a therapeutically effective amount of the cosmetic composition can be applied to the skin of a patient. The cosmetic composition of the present disclosure can be topically applied to the skin of a patient in any suitable manner. For example, the cosmetic composition can be transferred directly from a container to the target site of the patient's skin, applied with an applicator to the target site of the patient's skin, sprayed onto the target site of the patient's skin, and the like. In some examples, the cosmetic composition may be rubbed into the skin of the patient upon application. In embodiments in which the cosmetic composition is included as part of a delivery system such as a transdermal patch, the delivery system is applied to the skin of the patient such that the cosmetic composition is brought into contact with the skin via the layer(s) of the transdermal patch. Application of the cosmetic composition at a target area of a patient provides for transcutaneous delivery of the one or more cosmetic components to the target tissue in the target area.

This approach to delivery can be particularly effective in areas where a uniform application is desired. This approach to delivery may also be a preferred alternative to applications in which needle injection may be less desirable. Examples include crow's feet, perioral lines, extensive face and neck lines and general aging of the skin.

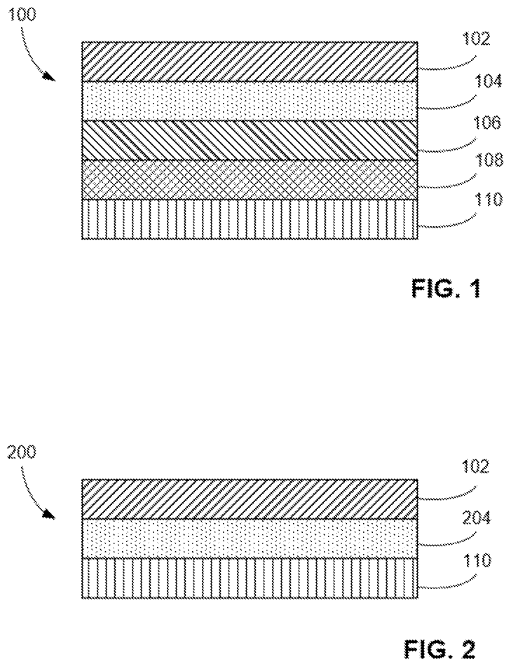

FIG. 1 is a schematic cross-sectional view showing layers of an exemplary transdermal patch 100. The transdermal patch 100 includes a backing layer 102, drug reservoir 104, rate-controlling membrane layer 106, and adhesive layer 108. The drug reservoir is disposed between the backing layer and the rate-controlling membrane layer. The adhesive layer is adjacent a surface of the rate-controlling membrane layer distal the drug reservoir. This transdermal patch 100 arrangement may also be referred to as a reservoir patch. A reservoir patch typically includes a reservoir of the drug where the reservoir is disposed between the backing layer and one or more permeable layers of material that that allows the drug to pass therethrough to contact the skin of a patient. A release liner may be attached to the adhesive layer 110.

The backing layer 102 may be made of any suitable material. The backing layer is typically made of plastic or other resilient material and may be impermeable to gas and/or liquid. Examples of the backing layer include papers such as impregnated papers, coated papers, plastic films such as polyester films, polyethylene films, polypropylene films, polyvinyl chloride films, polycarbonate films, polyurethane films and cellophane films, cloth bases such as non-woven cloths and woven cloths, and laminates thereof.

The drug reservoir 104 is a layer containing the cosmetic composition. The drug reservoir 104 may be defined by the shape of the backing layer 102. For example, the backing layer 102 may have a convex or cup shape that defines a volume between the surface of the backing layer 102 and the rate-controlling membrane layer 106 in which the cosmetic composition is disposed. In other embodiments, the cosmetic composition is simply sandwiched between the backing layer 102 and the rate-controlling membrane layer 106.

The rate-controlling membrane layer 106 is a typically made of a material that is permeable to the cosmetic composition. The selected permeability of the rate-controlling membrane layer 106 controls the rate at which the cosmetic composition is delivered to the patient. The rate-controlling membrane layer may be made of any suitable material. The rate-controlling membrane layer 106 may be a porous or non-porous layer. The rate-controlling membrane layer 106 may be a permeable film and/or foam. Exemplary materials include plastics such as polyester, polyethylene, polypropylene, polyvinyl chloride, polycarbonate, polyurethane and polyamide, cloth bases such as non-woven cloths, woven cloths and fabrics containing foams, polyester fibers, polyethylene fibers and polypropylene fibers.

The adhesive layer 108 may include any suitable adhesive. A wide variety of pharmaceutically-acceptable adhesive polymers can be used. Examples of adhesives which can be used in the patches include acrylic adhesives, polyacrylic adhesive polymers, acrylate copolymers (e.g., polyacrylate), silicone-based adhesives, polyisobutylene adhesive polymers, and combinations thereof. In some embodiments the adhesive layer 108 is a continuous layer. In other embodiments, the adhesive layer 108 is not a continuous layer (e.g., dots or other suitable pattern) or is provided as a grid pattern such that portions of the rate-controlling membrane layer 106 can be brought into contact with the skin of a patient upon application of the patch.

The release liner 110 may be appropriately selected to provide easy peeling from the adhesive layer. Exemplary release layer materials include polyethylene, polypropylene and polyester. Other exemplary release layers include paper. In some embodiments, the surface of the release liner 110 can be coated with silicone or a fluorocarbon.

In some embodiments, the transdermal patch 100 can be produced by applying the adhesive layer 108 onto the release liner 110, laminating and/or forming the rate-controlling membrane layer 106 on the surface of the adhesive layer 108 distal the release liner 110, and applying the cosmetic composition (drug reservoir 104) and backing layer 102 to the surface of the rate-controlling membrane layer 106 distal the adhesive layer 108. In some examples, the backing layer is formed into a desired shape (thereby forming the drug reservoir 104) and the cosmetic composition is placed inside the drug reservoir 104, and the assembly including the release liner 110, adhesive layer 108, and rate-controlling membrane layer 106 is brought into contact and laminated with the assembly including the backing layer 102 and cosmetic composition (drug reservoir 104) so that the drug reservoir 104 including the cosmetic composition is sandwiched between the backing layer 102 and the rate-controlling membrane layer 106. In other examples, the cosmetic composition is applied on the surface of the rate-controlling membrane layer 106 distal the adhesive layer 108, and the backing layer 102 is laminated on top of the cosmetic composition, thereby sandwiching the cosmetic composition (drug reservoir 104) between the backing layer 102 and the rate-controlling membrane layer 106.

FIG. 2 is a schematic cross-sectional view showing layers of another exemplary transdermal patch 100. The transdermal patch 100 includes a backing layer 102 and a drug and adhesive layer 204. In the example shown, the drug and adhesive layer 204 is adjacent the backing layer 102. This transdermal patch 200 may also be referred to as a matrix patch. In matrix patches, the drug and adhesive can be mixed. A release liner 110 may be attached to the drug and adhesive layer 204.

The backing layer 102 and release liner 110 may be made of the same materials as those described above with respect to transdermal patch 100.

The drug and adhesive layer 204 may be provided such that the cosmetic composition is provided in a matrix of adhesive. The adhesive of the drug and adhesive layer 204 may be any suitable adhesive such as the adhesives described with respect to transdermal patch 100. The adhesive matrix can contain varying amounts of the cosmetic composition. In some embodiments the cosmetic composition is present in the adhesive matrix in an amount of from 0.1 wt % to 60 wt %. In other embodiments the cosmetic composition is present in an amount from 0.1 wt % to 40 wt %. In other embodiments the cosmetic composition is present in an amount from 1 wt % to 30 wt %. In other embodiments the cosmetic composition is present in an amount from 1 wt % to 20 wt %. The drug and adhesive layer 204 can be produced by blending the cosmetic composition with the adhesive.

In some embodiments, the transdermal patch 200 can be produced by applying the drug and adhesive layer 204 onto the release liner 110, and laminating the backing layer 102 onto the surface of the drug and adhesive layer 204 distal the release liner 110. In other embodiments, the transdermal patch 200 can be produced by applying the drug and adhesive layer 204 onto the backing layer 102, and laminating the release liner 110 onto the surface of the drug and adhesive layer 204 distal the backing layer 102.

### EXAMPLES: COSMETIC COMPOSITION

For each of Examples 1-9, the ingredients are combined and mixed to form a cosmetic composition in liquid form. In each of the examples, the respective ingredients are mixed at room temperature (25° C.) in their respective amounts to provide a homogeneous mixture. The botulinum toxin was provided in 0.9% saline solution. The hyaluronic acid was provided in solution as a gel. The exosomes were provided in solution. For each example, the one or more rejuvenation cosmetic component was provided and DMSO and/or DMSO₂ was added to yield the vol% shown in Table 1.

TABLE 1
Examples 1-9

| | Amount (vol %) | | | | | | | | |
|---|---|---|---|---|---|---|---|---|---|
| Ingredient | Ex. 1 | Ex. 2 | Ex. 3 | Ex. 4 | Ex. 5 | Ex. 6 | Ex. 7 | Ex. 8 | Ex. 9 |
| dimethyl sulfoxide (DMSO) | 10 | 5 | | 10 | 10 | 10 | 10 | 10 | 10 |
| dimethyl sulfone (DMSO₂) | | 5 | 10 | | | | | | |
| botulinum toxin (2 mL, 100 unit) | 90 | 90 | 90 | 45 | 45 | 30 | | | |
| hyaluronic acid (2 mL, 20 mg/mL) | | | | 45 | | 30 | 90 | | 45 |
| exosomes (2 ml, 30 μg/mL) | | | | | 45 | 30 | | 90 | 45 |
| | 100 | 100 | 100 | 100 | 100 | 100 | 100 | 100 | 100 |

Examples 1-9 demonstrate exemplary compositions at which the solvent is present at 10 vol %. It will be understood that in other embodiments/examples, the amount of solvent relative to the one or more rejuvenation cosmetic components can be provided in other appropriate amounts. The following Examples provide other exemplary combinations.

For each of Examples 10-18, the ingredients are combined and mixed to form a cosmetic composition in liquid form. In each of the examples, the respective ingredients are mixed at room temperature (25° C.) in their respective amounts to provide a homogeneous mixture. For each example, the rejuvenation cosmetic component was provided and DMSO was added to yield the vol % shown in Table 2.

TABLE 2
Examples 10-18

| | Amount (vol %) | | | | | | | | |
|---|---|---|---|---|---|---|---|---|---|
| Ingredient | Ex. 10 | Ex. 11 | Ex. 12 | Ex. 13 | Ex. 14 | Ex. 15 | Ex. 16 | Ex. 17 | Ex. 18 |
| dimethyl sulfoxide (DMSO) | 5 | 15 | 20 | 25 | 30 | 35 | 40 | 45 | 50 |
| botulinum toxin (2 mL, 100 unit) | 95 | 80 | 80 | 75 | 70 | 65 | 60 | 55 | 50 |
| | 100 | 100 | 100 | 100 | 100 | 100 | 100 | 100 | 100 |

For each of Examples 19-27, the ingredients are combined and mixed to form a cosmetic composition in liquid form. In each of the examples, the respective ingredients are mixed at room temperature (25° C.) in their respective amounts to provide a homogeneous mixture. For each example, the rejuvenation cosmetic component was provided and DMSO was added to yield the vol% shown in Table 3.

TABLE 3
Examples 19-27

| | Amount (vol %) | | | | | | | | |
|---|---|---|---|---|---|---|---|---|---|
| Ingredient | Ex. 19 | Ex. 20 | Ex. 21 | Ex. 22 | Ex. 23 | Ex. 24 | Ex. 25 | Ex. 26 | Ex. 27 |
| dimethyl sulfoxide (DMSO) | 5 | 15 | 20 | 25 | 30 | 35 | 40 | 45 | 50 |
| hyaluronic acid (2 mL, 20 mg/mL) | 95 | 80 | 80 | 75 | 70 | 65 | 60 | 55 | 50 |
| | 100 | 100 | 100 | 100 | 100 | 100 | 100 | 100 | 100 |

For each of Examples 28-36, the ingredients are combined and mixed to form a cosmetic composition in liquid form. In each of the examples, the respective ingredients are mixed at room temperature (25° C.) in their respective amounts to provide a homogeneous mixture. For each example, the rejuvenation cosmetic component was provided and DMSO was added to yield the vol % shown in Table 4.

TABLE 4
Examples 28-36

| | Amount (vol %) | | | | | | | | |
|---|---|---|---|---|---|---|---|---|---|
| Ingredient | Ex. 28 | Ex. 29 | Ex. 30 | Ex. 31 | Ex. 32 | Ex. 33 | Ex. 34 | Ex. 35 | Ex. 36 |
| dimethyl sulfoxide (DMSO) | 5 | 15 | 20 | 25 | 30 | 35 | 40 | 45 | 50 |
| exosomes (2 ml, 30 μg/mL) | 95 | 80 | 80 | 75 | 70 | 65 | 60 | 55 | 50 |
| | 100 | 100 | 100 | 100 | 100 | 100 | 100 | 100 | 100 |

For each of Examples 37-45, the ingredients are combined and mixed to form a cosmetic composition in liquid form. In each of the examples, the respective ingredients are mixed at room temperature (25° C.) in their respective amounts to provide a homogeneous mixture. For each example, the rejuvenation cosmetic component was provided and DMSO was added to yield the vol % shown in Table 5.

TABLE 5
Examples 37-45

| | Amount (vol %) | | | | | | | | |
|---|---|---|---|---|---|---|---|---|---|
| Ingredient | Ex. 37 | Ex. 38 | Ex. 39 | Ex. 40 | Ex. 41 | Ex. 42 | Ex. 43 | Ex. 44 | Ex. 45 |
| dimethyl sulfoxide (DMSO) | 55 | 60 | 65 | 70 | 75 | 80 | 85 | 90 | 95 |
| botulinum toxin (2 mL, 100 unit) | 45 | 40 | 35 | 30 | 25 | 20 | 15 | 10 | 5 |
| | 100 | 100 | 100 | 100 | 100 | 100 | 100 | 100 | 100 |

For each of Examples 46-54, the ingredients are combined and mixed to form a cosmetic composition in liquid form. In each of the examples, the respective ingredients are mixed at room temperature (25° C.) in their respective amounts to provide a homogeneous mixture. For each example, the rejuvenation cosmetic component was provided and DMSO was added to yield the vol % shown in Table 6.

TABLE 6
Examples 46-54

| | Amount (vol %) | | | | | | | | |
|---|---|---|---|---|---|---|---|---|---|
| Ingredient | Ex. 46 | Ex. 47 | Ex. 48 | Ex. 49 | Ex. 50 | Ex. 51 | Ex. 52 | Ex. 53 | Ex. 54 |
| dimethyl sulfoxide (DMSO) | 55 | 60 | 65 | 70 | 75 | 80 | 85 | 90 | 95 |
| hyaluronic acid (2 mL, 20 mg/mL) | 45 | 40 | 35 | 30 | 25 | 20 | 15 | 10 | 5 |
| | 100 | 100 | 100 | 100 | 100 | 100 | 100 | 100 | 100 |

For each of Examples 55-63, the ingredients are combined and mixed to form a cosmetic composition in liquid form. In each of the examples, the respective ingredients are mixed at room temperature (25° C.) in their respective amounts to provide a homogeneous mixture. For each example, the rejuvenation cosmetic component was provided and DMSO was added to yield the vol % shown in Table 7.

TABLE 7
Examples 55-63

| | Amount (vol %) | | | | | | | | |
|---|---|---|---|---|---|---|---|---|---|
| Ingredient | Ex. 55 | Ex. 56 | Ex. 57 | Ex. 58 | Ex. 59 | Ex. 60 | Ex. 61 | Ex. 62 | Ex. 63 |
| dimethyl sulfoxide (DMSO) | 55 | 60 | 65 | 70 | 75 | 80 | 85 | 90 | 95 |
| exosomes (2 ml, 30 μg/mL) | 45 | 40 | 35 | 30 | 25 | 20 | 15 | 10 | 5 |
| | 100 | 100 | 100 | 100 | 100 | 100 | 100 | 100 | 100 |

In some examples, the cosmetic composition produced in any one of Examples 1-63 is applied to the skin of a patient. Application can be direct wherein the liquid is applied (e.g., poured or via an applicator) on the skin. Alternatively, application can be via a transdermal patch in wherein the liquid is applied to a carrier layer or contained in a drug reservoir of the delivery system, and the transdermal patch is applied to the skin.

It will be appreciated that the amount and concentration of each of the one or more rejuvenation cosmetic components can be varied as needed, depending on the size and area of the patient to be treated. Different parts of the body to be treated require different volumes and loadings for effective therapeutic treatment. For example, conventionally injection of botulinum toxin to the face of a patient can vary anywhere between 1-50 units depending on the area to be treated. Other areas of the body can require different of botulinum toxin. The botulinum toxin in the cosmetic composition can be provided in any suitable amount. In some examples, the botulinum toxin of the cosmetic composition is provided at 1 unit to 200 units. In other examples, the botulinum toxin of the cosmetic composition is provided at 1 unit to 100 units. In other examples, the botulinum toxin of the cosmetic composition is provided at 1 unit to 50 units. In some embodiments in which hyaluronic acid is included in the cosmetic composition, the hyaluronic acid can be provided in any suitable amount. In some examples, the hyaluronic acid is provided in an amount ranging from 0.1 mg/mL to 100 mg/mL. In other examples, the hyaluronic acid is provided in an amount ranging from 0.1 mg/mL to 50 mg/mL. In other examples, the hyaluronic acid is provided in an amount ranging from 1 mg/mL to 25 mg/mL. In other examples, the hyaluronic acid is provided in an amount ranging from 5 mg/mL to 20 mg/mL. In some embodiments in which exosomes is included in the cosmetic composition, the exosomes can be provided in any suitable amount. In some examples, the exosomes is provided in an amount ranging from 0.1 μg/mL to 1000 μg/mL. In other examples, the exosomes is provided in an amount ranging from 1 μg/mL to 500 μg/mL. In other examples, the exosomes is provided in an amount ranging from 1 μg/mL to 100 μg/mL. In other examples, the exosomes is provided in an amount ranging from 10 μg/mL to 50 μg/mL.

In some examples, the composition produced in any one of the Examples 1-63 is further combined with one or more additional components (e.g., water, one or more oils, one or more water-soluble or water-swelling polymers, one or more additional components, one or more oil-soluble gelling agents, one or more clay minerals modified with organic compounds, one or more moisture retention agents, one or more antiseptics, one or more anti-bacterial agents, one or more fragrances, one or more salts, one or more antioxidants, one or more pH regulators, one or more chelating agents, one or more refreshing agents, one or more anti-inflammatory agents, one or more skin beautifying components such as skin whitener, cell activator, rough dry skin improver, blood circulation promoter, skin astringent and anti-seborrheic agent, one or more vitamins, one or more amino acids, one or more nucleic acids, one or more hormones, and one or more clathrate compounds.). Combination with the one or more additional components may allow for the resultant cosmetic composition to be provided in a suitable form (e.g., liquid, emulsion, cream, paste, gel, mousse, and the like).

Application can be direct wherein the liquid is applied (e.g., poured or via an applicator) on the skin. Alternatively, application can be via a transdermal patch in wherein the liquid is applied to a carrier layer and the transdermal patch is applied to the skin.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A cosmetic composition, comprising:
one or more solvents, the one or more solvents comprising dimethyl sulfoxide and/or dimethyl sulfone; and
one or more rejuvenation cosmetic components, the one or more rejuvenation cosmetic components comprising botulinum toxin, hyaluronic acid, and/or exosomes.

2. The cosmetic composition of claim 1, wherein the one or more solvents is dimethyl sulfoxide.

3. The cosmetic composition of claim 1, wherein the one or more solvents is dimethyl sulfone.

4. The cosmetic composition of claim 1, wherein the one or more solvents is a combination of dimethyl sulfoxide and dimethyl sulfone.

5. The cosmetic composition of claim 1, wherein a total amount of the one or more solvents in the cosmetic composition ranges from 0.1 vol % to 99.9 vol %.

6. The cosmetic composition of claim 1, wherein a total amount of the one or more solvents in the cosmetic composition ranges from 0.1 vol % to 50 vol %.

7. The cosmetic composition of claim 1, wherein the one or more rejuvenation cosmetic components comprises botulinum toxin.

8. The cosmetic composition of claim 1, wherein the one or more rejuvenation cosmetic components comprises hyaluronic acid.

9. The cosmetic composition of claim 1, wherein the one or more rejuvenation cosmetic components comprises exosomes.

10. The cosmetic composition of claim 1, wherein the one or more rejuvenation cosmetic components comprises a combination of botulinum toxin and hyaluronic acid.

11. The cosmetic composition of claim 1, wherein the one or more rejuvenation cosmetic components comprises a combination of botulinum toxin and exosomes.

12. The cosmetic composition of claim 1, wherein the one or more rejuvenation cosmetic components comprises a combination of hyaluronic acid and exosomes.

13. The cosmetic composition of claim 1, further comprising water.

14. The cosmetic composition of claim 1, further comprising one or more oils.

15. The cosmetic composition of claim 1, further comprising one or more water-soluble or water-swelling polymers.

16. The cosmetic composition of claim 1, wherein the cosmetic composition is a liquid composition in the form of a cream, paste, gel, or mousse.

17. A delivery system, comprising:
a transdermal patch comprising the cosmetic composition of claim 1.

18. The delivery system of claim 17, wherein the transdermal patch comprises:
a backing layer;
a rate-controlling membrane layer;
a drug reservoir disposed between the backing layer and the rate-controlling membrane layer, the drug reservoir containing the cosmetic composition; and
an adhesive layer adjacent a surface of the rate-controlling membrane layer distal the drug reservoir.

19. The delivery system of claim 17, wherein the transdermal patch comprises:
a backing layer; and
a drug and adhesive layer adjacent the backing layer, the drug and adhesive layer comprising a matrix of adhesive in which the cosmetic composition is disposed.

20. A method of transcutaneous delivery of one or more rejuvenation cosmetic components, comprising topically applying a therapeutically effective amount of the cosmetic composition of claim 1 to the skin of a patient.
